(19) United States
(12) Patent Application Publication
(10) Pub. No.: US 2023/0322879 A1
(43) Pub. Date: Oct. 12, 2023

(54) CYTOKINE-BASED MULTI-EPITOPE PROTEIN FOR BINDING TO CCR7-POSITIVE CELLS

(71) Applicants: Maria Beihaghi, Mashhad (IR); Hasan Marashi, Mashhad (IR)

(72) Inventors: Maria Beihaghi, Mashhad (IR); Hasan Marashi, Mashhad (IR)

(73) Assignee: Ferdowsi University of Mashhad, Mashhad (IR)

(21) Appl. No.: 18/073,599

(22) Filed: Dec. 2, 2022

Related U.S. Application Data
(63) Continuation-in-part of application No. PCT/IB2021/060087, filed on Nov. 1, 2021.

(51) Int. Cl. C07K 14/535; C07K 14/545

(52) U.S. Cl. CPC C07K 14/535; C07K 2319/03; C07K 2319/02; C07K 14/545

(57) ABSTRACT

A cytokine-based multi-epitope protein for binding to CC-chemokine receptor type 7 (CCR7)-positive cells, including immunomodulatory molecules. The immunomodulatory molecules include a truncated granulocyte-macrophage colony-stimulating factor (GM-CSF), truncated chemokines, a truncated interleukin 1 beta (IL-1β), and a chemokine secretory signal peptide. The truncated chemokines include a truncated CC-chemokine ligand-19 (CCL19) and a truncated CC-chemokine ligand-21 (CCL21). Each of the truncated chemokines includes a respective DCCL motif, a respective putative receptor binding cleft, and a respective putative glycosaminoglycan binding site.

### CROSS-REFERENCE TO RELATED APPLICATION

This application is a continuation-in-part of International Patent Application PCT/IB2021/060087, filed on Nov. 1, 2021, and entitled “CYTOKINE-BASED MULTI-EPITOPE PROTEIN FOR BINDING TO CCR7-POSITIVE CELLS,” which takes priority from U.S. Provisional Patent Application Ser. No. 63/208,004, filed on Jun. 8, 2021, and entitled “RECOMBINANT MULTI-EPITOPE PROTEIN BASED ON CYTOKINE GENES THAT BIND TO THE CCR7 RECEPTOR,” which are both incorporated herein by reference in their entirety.

### STATEMENT REGARDING SEQUENCE LISTING

The Sequence Listing associated with this application is provided in XML format in lieu of a paper copy and is hereby incorporated by reference into the specification. The name of the XML file containing the Sequence Listing is (IP 7214) Beihaghi Sequence Listing.xml. The XML file is 10 KB, was created on Jun. 24, 2023, and is being submitted electronically via Patent Center.

### TECHNICAL FIELD

The present disclosure generally relates to cytokine-based proteins, particularly to cytokine-based multi-epitope proteins, and more particularly to cytokine-based multi-epitope proteins for simultaneous activation of innate and adaptive immune systems.

### BACKGROUND

Generally, the primary methods used for treating cancer, like chemotherapy, cause adverse side effects such as hair loss, nausea, and weakness. These complications occur because cancer treatments tend to aim to kill cancer cells, and in this fight, some of the body's healthy cells are also damaged, which weakens the immune system. As a result, utilizing a compound that can strengthen and improve the immune system without any side effects before cancer treatments like chemotherapy is of interest. In addition, many of the drugs suggested for the treatment of viral diseases, such as AIDS and coronavirus, produce potent antibodies in the body, which can stimulate and improve the immune system in people with the disease.

Currently, there are different animal-derived drugs as immunotherapy agents based on CCR7 receptor to strengthen the patient's immune system. However, these immunotherapy agents have several limitations. For example, some of them only affects T-lymphocytes and CTLs and reduces regulatory T-cell expression, but it has no role in activating B-lymphocytes, macrophages, and neutrophils.

Therefore, there is a need to produce a multi-epitope drug based on cytokine genes with high immunogenicity and specificity to destroy cancer cells and prevent viral diseases such as AIDS and COVID-19 without any side effects. Also, there is a need for producing recombinant cytokine-based multi-epitope drugs that may simultaneously stimulate a patient's innate and adaptive immune systems without causing an autoimmune reaction. Furthermore, there is a need for a helpful biomarker for cancer screening and prognosis tests.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary cytokine-based multi-epitope protein for binding to CC-chemokine receptor type 7 (CCR7)-positive cells. In an exemplary embodiment, an exemplary cytokine-based multi-epitope may include immunomodulatory molecules. In an exemplary embodiment, the immunomodulatory molecules may include a truncated granulocyte-macrophage colony-stimulating factor (GM-CSF), truncated chemokines, a truncated interleukin 1 beta (IL-1β), and a chemokine secretory signal peptide. In an exemplary embodiment, the truncated chemokines may include a truncated CC-chemokine ligand-19 (CCL19) and a truncated CC-chemokine ligand-21 (CCL21). In an exemplary embodiment, each of the truncated chemokines may include a respective DCCL motif, a respective putative receptor binding cleft, and a respective putative glycosaminoglycan binding site.

In an exemplary embodiment, the truncated GM-CSF may be connected to the CCL19 through a helical linker. In an exemplary embodiment, the truncated CCL19 may be connected to the CCL21 through a furine protease-sensitive linker. In an exemplary embodiment, the truncated CCL21 may be connected to the truncated IL-1β through a cathepsin-sensitive linker. In an exemplary embodiment, the truncated IL-1β may be connected to the chemokine secretory signal peptide directly.

In an exemplary embodiment, an exemplary cytokine-based multi-epitope protein may include SEQ ID NO: 1. In an exemplary embodiment, an exemplary cytokine-based multi-epitope protein may include SEQ ID NO: 1 encoded by SEQ ID NO: 2. In an exemplary embodiment, the truncated GM-CSF may include SEQ ID NO: 3. In an exemplary embodiment, the truncated CCL19 may include SEQ ID NO: 4. In an exemplary embodiment, the truncated CCL21 may include SEQ ID NO: 5. In an exemplary embodiment, the truncated IL-1β may include SEQ ID NO: 6. In an exemplary embodiment, the chemokine secretory signal peptide may include rat KC chemokine. In an exemplary embodiment, the rat chemokine KC may include SEQ ID NO: 7.

In an exemplary embodiment, the CCR7-positive cells may include at least one of CCR7-positive breast cancer cells, CCR7-positive lung cancer cells, monocytes, T lymphocytes, B lymphocytes, natural killer (NK) cells, and dendritic cells (DCs). In an exemplary embodiment, an exemplary cytokine-based multi-epitope protein may have a molecular weight between about 60 kDa and about 65 kDa. In an exemplary embodiment, exemplary cytokine-based multi-epitope protein may further include a purification tag, including at least one of a polyhistidine tag and a glutathione S-transferase (GST) tag.

In an exemplary embodiment, an exemplary cytokine-based multi-epitope protein may be a hydrophilic protein with a grand average of hydropathicity index (GRAVY) of about 1.25. In an exemplary embodiment, an exemplary cytokine-based multi-epitope protein may be a thermostable protein with an aliphatic index of 84.57. In an exemplary embodiment, an exemplary cytokine-based multi-epitope protein may be transmembrane. In an exemplary embodiment, an exemplary cytokine-based multi-epitope protein may have a non-allergenicity index of more than 98%. In an exemplary embodiment, an exemplary cytokine-based multi-epitope protein may be a thermostable protein with an instability index of about 30.5. In an exemplary embodiment, an exemplary cytokine-based multi-epitope protein may have an in-vitro half-life of less than about 30 hours in mammalian reticulocytes, less than about 20 hours in yeasts, and less than about 10 hours in Escherichia coli cells.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates a three-dimensional (3D) structure of an exemplary cytokine-based multi-epitope protein, consistent with one or more exemplary embodiments of the present disclosure.

FIG. 2 illustrates root deviation of the mean squares (RMSD) changes related to exemplary cytokine-based multi-epitope protein during 100 nm of molecular dynamics simulation, consistent with one or more exemplary embodiments of the present disclosure.

FIG. 3 illustrates changes in the radius of Gyration of an exemplary cytokine-based multi-epitope protein during 100 nm of molecular dynamics simulation, consistent with one or more exemplary embodiments of the present disclosure.

FIG. 4A illustrates the 3D structure of an exemplary multi-epitope protein before 100 nanoseconds of MD simulation, consistent with one or more exemplary embodiments of the present disclosure.

FIG. 4B illustrates the 3D structure of an exemplary cytokine-based multi-epitope protein after 100 nanoseconds of MD simulation, consistent with one or more exemplary embodiments of the present disclosure.

FIG. 5 illustrates a molecular docking complex between an exemplary cytokine-based multi-epitope protein and CCR7, consistent with one or more exemplary embodiments of the present disclosure.

FIG. 6 illustrates changes in the RMSD diagram of the exemplary cytokine-based multi-epitope protein in the CCR7 binding state during MD simulations, consistent with one or more exemplary embodiments of the present disclosure.

FIG. 7 illustrates relative expression of CCL21 and CCL19 epitopes in E. coli compared to the beta-actin gene as a housekeeping gene using real-time PCR, consistent with one or more exemplary embodiments of the present disclosure.

FIG. 8A illustrates an image of SDS-PAGE of exemplary cytokine-based multi-epitope protein, including well 1: total protein extracted from transgenic E. coli, well 2: fraction flowing from the column, well 3: protein ladder, well 4: fractions resulting from washing with 500 mM imidazole buffer solution, well 5: fractions resulting from rinsing with 100 mM imidazole buffer solution, well 6: fractions resulting from elution with 250 mM imidazole buffer solution, well 7: Negative control (total protein extracted from non-transgenic E. coli), consistent with one or more exemplary embodiments of the present disclosure.

FIG. 8B illustrates Western blot results of an exemplary cytokine-based multi-epitope protein including well M: a molecular marker of protein, well 1 and well 2: exemplary cytokine-based multi-epitope protein, well 3: commercial CC121 antigen, and well 4: Negative control (total protein extracted from non-transgenic E. coli), consistent with one or more exemplary embodiments of the present disclosure.

FIG. 9A illustrates a standard curve of commercial antigen CCL21, consistent with one or more exemplary embodiments of the present disclosure.

FIG. 9B illustrates quantitative measurement of exemplary cytokine-based multi-epitope protein using ELISA, including a transgenic bacteria (TG) expressing the exemplary cytokine-based multi-epitope protein, non-transgenic bacteria as a negative control (NC), and bovine serum albumin (BSA) (blank), consistent with one or more exemplary embodiments of the present disclosure.

FIG. 10 illustrates Fourier-transform infrared spectroscopy (FTIR) spectra of the exemplary cytokine-based multi-epitope protein (A) and commercial CCL21 (B), consistent with one or more exemplary embodiments of the present disclosure.

FIG. 11 illustrates relative gene expressions in different groups, including DMEM medium without PBMC cells as a negative control (NC), expression of CCR7 (A), CCL19 (B), and CCL21 (C) in PBMC cells of cancer samples, expression of CCR7 (D), CCL19 (E), and CCL21 (F) in PBMC cells of healthy samples, expression of CCR7 (G), CCL19 (H), and CCL21 (I) in PBMC cells of cancer samples treated with cytokine-based multi-epitope protein, expression of CCR7 (J), CCL19 (K), and CCL21 (L) in PBMC cells of cancer samples treated with commercial CCL21, consistent with one or more exemplary embodiments of the present disclosure.

FIG. 12A illustrates an evaluation of the toxicity of the exemplary cytokine-based multi-epitope protein on CCR7⁺ MCF7 cancer cells at 24, 48, and 72 hours after incubation using the MTT assay compared to the DMEM medium as a negative control group at different concentrations of 2.5, 5, 7.5, and 10 μg/ml, consistent with one or more exemplary embodiments of the present disclosure.

FIG. 12B illustrates an evaluation of the toxicity of a commercial CCL21 antigen on MCF7 cancer cells at 24, 48, and 72 hours after incubation using the MTT assay compared to the negative control group at different concentrations of 2.5, 5, 7.5, and 10 μg/ml, consistent with one or more exemplary embodiments of the present disclosure.

FIG. 13 illustrates the effect of the exemplary cytokine-based multi-epitope protein, commercial CCL21 protein, and DMEM medium as a negative control on the migration of MCF7 cancer cells at different times (24, 48, and 72 hours after wound induction), consistent with one or more exemplary embodiments of the present disclosure.

FIG. 14 illustrates a comparison between chemokine (CK) and chemotaxis (CT) properties: A) migration of PBMC cells to the exemplary cytokine-based multi-epitope protein containing CCL21 and CCL19 epitopes (CK), B) migration of PBMC to 10% FBS (CT), consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

The present disclosure describes an exemplary cytokine-based multi-epitope protein binding to CC-chemokine receptor type 7 (CCR7)-positive cells to simultaneously activate innate and adaptive immune systems. In an exemplary embodiment, an exemplary cytokine-based multi-epitope protein may bind to and activate the CCR7-positive (CCR7⁺) immune cells, including T-cell lymphocytes, B-cell lymphocytes, natural killer (NK) cells, dendritic cells (DCs), macrophages, and neutrophils. In an exemplary embodiment, an exemplary cytokine-based multi-epitope protein may also bind to CCR7⁺ cancer cells, including at least one of CCR7⁺ breast cancer cells and CCR7⁺ lung cancer cells.

In an exemplary embodiment, an exemplary cytokine-based multi-epitope may include immunomodulatory molecules. In an exemplary embodiment, the immunomodulatory molecules may include a truncated granulocyte-macrophage colony-stimulating factor (GM-CSF), truncated chemokines, a truncated interleukin 1 beta (IL-1β), and a chemokine secretory signal peptide. In an exemplary embodiment, the truncated chemokines may include a truncated CC-chemokine ligand-19 (CCL19) and a truncated CC-chemokine ligand-21 (CCL21). In an exemplary embodiment, each of the truncated chemokines may include a respective DCCL motif, a respective putative receptor binding cleft, and a respective putative glycosaminoglycan binding site.

In an exemplary embodiment, exemplary cytokine-based multi-epitope protein may increase the body's immunity against cancer and several viral diseases by increasing CD8⁺ T lymphocytes and activating various immune cells, such as neutrophiles, T lymphocytes, and B lymphocytes. In an exemplary embodiment, CCL21 and CCL19 epitopes of an exemplary cytokine-based multi-epitope protein may specifically bind to the CCR7 receptor, which is found on many cancer and tumor cells, and naïve T-cells and they also may chemoattract T-lymphocytes and DCs; as a result, the exemplary cytokine-based multi-epitope protein may have antitumor properties.

In an exemplary embodiment, an exemplary cytokine-based multi-epitope protein may be used for prognosis of tumorigenesis by binding of an exemplary cytokine-based multi-epitope protein to the cancer cells with higher affinity compared to the healthy cells. In an exemplary embodiment, an exemplary cytokine-based multi-epitope protein may bind to the cancer cells with higher affinity compared to the healthy cells due to presence of more CCR7 receptors on the cancer cells such as MCF7 cells of breast cancer than the healthy cells. For example, an exemplary cytokine-based multi-epitope protein may increase CD8⁺ T cells, leading to better detection and reduction of viral infections progressions, such as human immunodeficiency viruses (HIV) and coronavirus disease 2019 (COVID-19) infection. In an exemplary embodiment, an exemplary cytokine-based multi-epitope protein may effectively treat AIDS since the CCL21 epitope of the exemplary cytokine-based multi-epitope protein may occupy CCR7 receptors on the surface of CD4⁺ T cells and prevent HIV attachment.

In an exemplary embodiment, the IL-Iβ epitope of an exemplary cytokine-based multi-epitope protein may also involve cellular activities, such as neutrophil activation, T- and B-lymphocyte cell production, antibody production, and fibroblast proliferation. In an exemplary embodiment, granulocyte-macrophage colony-stimulating factor (GM-CSF) epitope of an exemplary cytokine-based multi-epitope protein as one of the growth factors of white blood cells (WBC) may stimulate stem cells to produce granulocytes and monocytes. Additionally, the exemplary cytokine-based multi-epitope protein may include specific sites for disulfide bonds and linkers to stabilize and activate the exemplary cytokine-based multi-epitope protein.

In an exemplary embodiment, the truncated GM-CSF may be connected to the CCL19 through a helical linker. In an exemplary embodiment, the truncated CCL19 may be connected to the CCL21 through a furine protease-sensitive linker. In an exemplary embodiment, the truncated CCL21 may be connected to the truncated IL-1β through a cathepsin-sensitive linker. In an exemplary embodiment, the truncated IL-1β may be connected to the chemokine secretory signal peptide directly. In an exemplary embodiment, an exemplary cytokine-based multi-epitope protein may include SEQ ID NO: 1. In an exemplary embodiment, an exemplary cytokine-based multi-epitope protein may include SEQ ID NO: 1 encoded by SEQ ID NO: 2.

In an exemplary embodiment, the truncated GM-CSF may include SEQ ID NO: 3. In an exemplary embodiment, the truncated CCL19 may include SEQ ID NO: 4. In an exemplary embodiment, the truncated CCL21 may include SEQ ID NO: 5. In an exemplary embodiment, the truncated IL-1β may include SEQ ID NO: 6. In an exemplary embodiment, the chemokine secretory signal peptide may include rat KC chemokine. In an exemplary embodiment, the rat chemokine KC may include SEQ ID NO: 7. In an exemplary embodiment, exemplary cytokine-based multi-epitope protein may further include a purification tag, including at least one of a polyhistidine tag and a glutathione S-transferase (GST) tag.

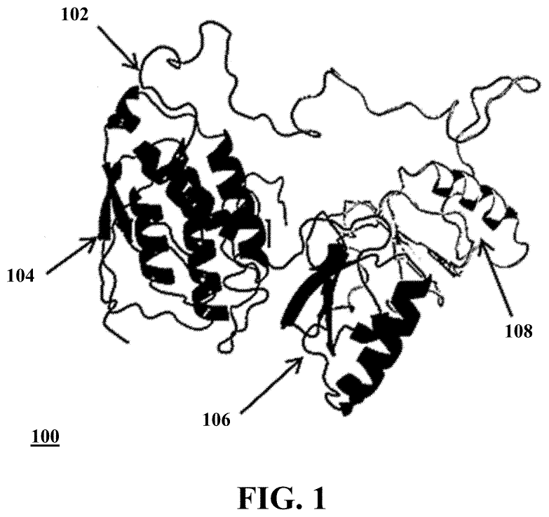

FIG. 1 illustrates a three-dimensional (3D) structure 100 of an exemplary cytokine-based multi-epitope protein, consistent with one or more exemplary embodiments of the present disclosure. Referring to FIG. 1, an exemplary cytokine-based multi-epitope protein may include different epitopes of different proteins, including IL-1β and signal peptide 102, GM-CSF 104, CCL19 106, and CCL21 108.

In an exemplary embodiment, an exemplary cytokine-based multi-epitope protein may have a molecular weight between about 60 kDa and about 65 kDa. In an exemplary embodiment, an exemplary cytokine-based multi-epitope protein may be a hydrophilic protein with a grand average of hydropathicity index (GRAVY) of about 1.25. In an exemplary embodiment, an exemplary cytokine-based multi-epitope protein may be a thermostable protein with an aliphatic index of 84.57. In an exemplary embodiment, an exemplary cytokine-based multi-epitope protein may be transmembrane.

In an exemplary embodiment, an exemplary cytokine-based multi-epitope protein may have a non-allergenicity index of more than 98%. In an exemplary embodiment, an exemplary cytokine-based multi-epitope protein may be a thermostable protein with an instability index of about 30.5. In an exemplary embodiment, an exemplary cytokine-based multi-epitope protein may have an in-vitro half-life of less than about 30 hours in mammalian reticulocytes, less than about 20 hours in yeasts, and less than about 10 hours in Escherichia coli cells.

In an exemplary embodiment, an exemplary cytokine-based multi-epitope protein may utilize CCL19, IL-Iβ, and GM-CSF as potent adjuvants for treating breast cancer, especially in combination with HER2/neu gene expression and the TH1 immune response. In an exemplary embodiment, an exemplary cytokine-based multi-epitope protein may bind specifically to an exemplary CCR7 receptor and may have antitumor properties. In an exemplary embodiment, an exemplary cytokine-based multi-epitope protein may be used as a biomarker for cancer screening and prognosis tests by detecting CCR7⁺ cancer cells through binding to CCR7 receptors of cancer cells in-vitro or in-vivo. In an exemplary embodiment, an exemplary cytokine-based multi-epitope protein may be used to identify patients whose tumors are more likely to progress and recur and may be used to immunize patients before chemotherapy and aggressive treatments by activating and stimulating the immune cells of adaptive and innate immune systems.

In an exemplary embodiment, an exemplary cytokine-based multi-epitope protein may reduce the progression of viral diseases, such as HIV and COVID-19. In an exemplary embodiment, exemplary cytokine-based multi-epitope protein may be used to improve a patient's immune system before chemotherapy to reduce the side effects by stimulating patient's immune system which leads for production of more immune cells. In an exemplary embodiment, exemplary cytokine-based multi-epitope protein may be used as a vaccine for increasing immunity against cancer and viral diseases with low cost and without any significant complication by activating the CCR7-positive (CCR7⁺) immune cells, including T-cell lymphocytes, B-cell lymphocytes, natural killer (NK) cells, dendritic cells (DCs), macrophages, and neutrophils. In an exemplary embodiment, the exemplary cytokine-based multi-epitope protein may enhance immune activation of cells effective to recognize and act against cancer cells by increasing and activating anti-tumor cells, such as lymphocytes and neutrophiles. In an exemplary embodiment, the exemplary cytokine-based multi-epitope protein may have lethality and anti-metastatic effect against cancer cells because of binding to CCR7 which is more on the surface of cancer cells in comparison with the healthy cells.

### EXAMPLES

### Example 1: Design and In-Silico Analysis of an Exemplary Cytokine-Based Multi-Epitope Protein

In this example, an exemplary cytokine-based multi-epitope protein (SEQ ID NO: 1) including different epitopes of human proteins CCL21, CCL19, IL-Iβ, and GM-CSF was designed. Firstly, candidate epitopes and linkers between them were selected for in-silico designing of the gene construct. Selection of the epitopes was based on their ability to activate both innate and adaptive immune systems by binding to major histocompatibility complex II and I (MHCII/MHCI) and CCR7 receptor, which is present on different immune cells, including T helper lymphocytes (THL), cytotoxic T lymphocytes (CTL) and B lymphocytes. The CCL21 and CCL19 sequences selected epitopes had a respective DCCL motif, a respective domain binding to the CCR7 receptor, a respective Pan HLA DR-binding epitope (PADRE) peptide sequence and a respective putative glycosaminoglycan binding site that covers more than 90% of the HLA alleles.

CCL21 and CC119 are chemokines that control cell trafficking and are involved in numerous pathologic and inflammatory conditions, and it is endocytosed with its receptor (CCR7), via both MEW class I and II processing pathways to induce CD8⁺ and CD4⁺ T-cell responses. These data suggest that the exemplary cytokine-based multi-epitope protein may be taken up, processed, and presented by APCs after binding and internalization through the CCR7 receptor. CCR7 facilitates the uptake and processing of tumor antigens to induce efficient CD4⁺ T-cell responses both in-vitro and in-vivo using the MEW class II antigen processing pathway. Since the selected epitopes of CCL21 and CCL19 have a half-maximal inhibitory concentration (IC₅₀) less than 50, they may bind to both MHCI and MHCII molecules.

In addition, a part of selected epitopes of IL-1β is involved in inflammatory and immune responses and has high adjuvant effects. While the selected epitope of GMCSF adjuvant covers more than 90% of HLA alleles and may only bind to MHCI molecules, it does not affect MHCII molecules since its IC₅₀ was greater than 50. By binding the exemplary cytokine-based multi-epitope protein to MHCI and MHCII molecules in T-cells and CCR7 receptors, the exemplary cytokine-based multi-epitope protein may produce anti-metastatic and cytotoxicity effects on cancer cell lines and chemotactic response in lymphocyte cells. In an exemplary embodiment, the exemplary cytokine-based multi-epitope protein may be recognized as endogenous chemokine via its CTL cell epitopes that may bind to MHCI. Also, binding the exemplary cytokine-based multi-epitope protein to MEW II may occur via the exogenous pathway of antigen presentation, which activates T helpers.

The gene construct also contained rat chemokine KC as a signal peptide and a polyhistidine tag for purification of the gene construct. In the gene construct, the GM-CSF was connected to the CCL19 through a helical linker (EAAAK), a beta-defensins that reduces interaction with other recombinant protein domains. The CCL19 was connected to the CCL21 through a furine protease-sensitive linker (RRVR). Also, the CCL21 was connected to the truncated IL-1β through a cathepsin B-sensitive linker (GPGPG). The IL-1β was directly connected to the rat KC chemokine without a linker. Moreover, the rat KC chemokine was connected to the polyhistidine tag (6×His tag) through an HIV protease-sensitive linker (RVLAEA).

After designing the exemplary cytokine-based multi-epitope protein, its physicochemical characteristics were determined using in-silico tools. The exemplary cytokine-based multi-epitope protein has an instability index of about 30.5, which indicates the stability of the recombinant multi-epitope protein. Also, the aliphatic index of the exemplary cytokine-based multi-epitope protein is about 84.57, which indicates the temperature resistance of the exemplary cytokine-based multi-epitope protein. Moreover, the grand average of hydropathicity of the exemplary cytokine-based multi-epitope protein is about −1.25, which indicates that the vaccine is hydrophilic. The exemplary cytokine-based multi-epitope protein has a solubility index of about 84.3%.

The in-vitro half-life of the exemplary cytokine-based multi-epitope protein is less than about 30 hours in mammalian cells, less than about 20 hours in yeasts, and less than about 10 hours in Escherichia coli. Furthermore, the allergenicity of the exemplary cytokine-based multi-epitope protein was evaluated, and it was shown that the exemplary cytokine-based multi-epitope protein has 98% non-allergenicity. Therefore, the exemplary cytokine-based multi-epitope protein may be used for treatment purposes.

### Example 2: Molecular Dynamic Simulation of an Exemplary Cytokine-Based Multi-Epitope Protein

Observation of changes in the orientation of different amino acids over time is feasible by using molecular dynamic simulation. In molecular dynamic (MD) simulation method, each atom has dynamics and motion, which causes movement in the structure of the whole protein, which may optimize the 3D structure of the recombinant modeling protein. Motion is the main factor in the function of biological macromolecules and, as a result, has created the “dynamic relationship between activities” approach. Dynamics are the main factors in the stability of biological systems, including the function of enzymes, drug binding, membrane formation, and other biological processes. A change in motion causes a change in structure and thus a change in protein function. Motion may eliminate a series of interactions in the structure of proteins and thus the formation of new interactions, which may optimize the structure of the proteins.

In this example, molecular dynamic simulation of an exemplary cytokine-based multi-epitope protein was done. Initially, since the 3D structure of an exemplary cytokine-based multi-epitope protein was not available, a comparative modeling method (homology modeling) was used to create the 3D structure of the exemplary cytokine-based multi-epitope protein. Comparative modeling refers to constructing an atomic-resolution model of a protein from its amino acid sequence and an experimental three-dimensional structure of a related homologous protein. In this modeling method, 3D structures with sequences very similar to the target sequence are used as the model. Ten models were produced using the comparative modeling method, and one with the most stability, interaction, and connectivity between the epitopes of the exemplary cytokine-based multi-epitope protein was selected as the best model (FIG. 1).

After 3D modeling of the target recombinant protein sequence, the molecular dynamics (MD) simulation method was used to optimize the modeled structure. A series of analyses are also used to evaluate the protein's 3D structure stability during the simulation.

The MD simulation was performed using a software. Input structures were prepared with ff99SB force field. The correct hydrogen status of histidine amino acids was defined for all proteins, histidine amino acids in the structure, formations, and disulfide bonds (if any) of the enzyme. The surface charge of the structure was neutralized by adding chlorine ions. The protein was placed in a layer of 8-angstrom thick TIP3P water molecules inside an octahedron box using gmx solvate software. Reduction of energy on the structures was achieved with 50,000 steps by the steepest descent method to eliminate van der Waals interactions and hydrogen bonds between water molecules and the complex.

After that, the system temperature was gradually increased from 0 to 310 K for 200 pc in constant volume, and then the system was equilibrated at constant pressure for 200 pc. Molecular dynamics simulations were performed at 37° C. for 100 nanoseconds. Non-bonded interactions with 10-angstrom intervals were calculated by the PME method. The SHAKE algorithm, which is known for satisfying a bond geometry constraint during molecular dynamics simulations, was also used to limit the bonds involved in the hydrogen atom and increase computational speed. Finally, the simulation information was stored at 0.4 pc intervals for analysis.

The root deviation of the mean squares (RMSD) between the structures created during the MD simulation in the time dimension is used to analyze the structural stability of the protein. Therefore, the RMSD changes related to alpha carbon atoms of the protein during the simulation time (100 nm) relative to the original structure were calculated and extracted.

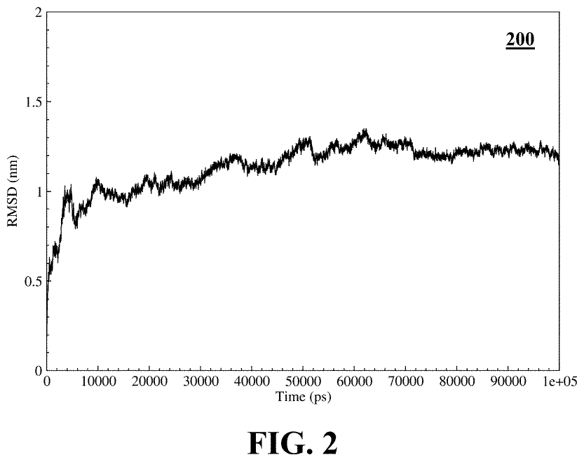

FIG. 2 illustrates diagram 200 which illustrates RMSD changes related to exemplary cytokine-based multi-epitope protein during 100 nanoseconds of molecular dynamics simulation, consistent with one or more exemplary embodiments of the present disclosure. Referring to FIG. 2, at the beginning of the MD simulation, the RMSD diagram shows an uptrend. In the first 10 nanoseconds of the simulation, the slope of the increase in RMSD is so rapid that after about 10,000 picoseconds (ps), the RMSD value reaches 1 nm, but the increase slows down; so that at 60,000 ps, the RMSD value is equal to 1.25 nanometer. After 60,000 ps, the RMSD value decreased slightly and reached 1.1 nm at 70,000 picoseconds, and remained constant at the end of the simulation, indicating the stability of the protein structure at the end of the simulation. It's known in the art that if a protein is stable, the RMSD value, after an initial increase, should fluctuate around a constant value.

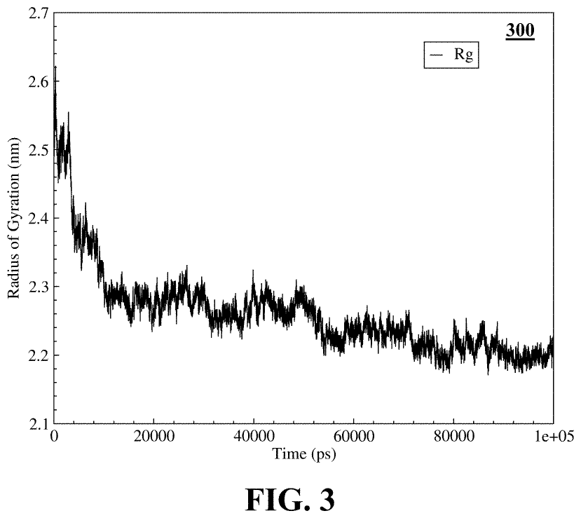

The radius of Gyration is one of the critical parameters in studying changes in protein size during the MD simulation. The lower the radius of Gyration during MD simulation, the more compact the protein. Conversely, as the radius of Gyration increases, the size of the protein increases too. FIG. 3 illustrates diagram 300 which illustrates changes in the radius of Gyration of an exemplary cytokine-based multi-epitope protein during 100 nanoseconds of molecular dynamics simulation, consistent with one or more exemplary embodiments of the present disclosure.

Referring to FIG. 3, the value of the radius of Gyration of an exemplary cytokine-based protein equals about 2.62 nm at the beginning of the MD simulation. However, as the simulation continues, the value of the radius drops rapidly and reaches 2.29 nm in about 10,000 picoseconds. It's known in the art that if the radius of Gyration of a protein increases during the MD simulation, it may indicate denaturation and instability of the protein. From this time onward, the change in the slope of the radius of Gyration slows down, such that at 80,000 picoseconds, the value is equal to about 2.21 nm, after which it remains constant until the end of the simulation, indicating stability.

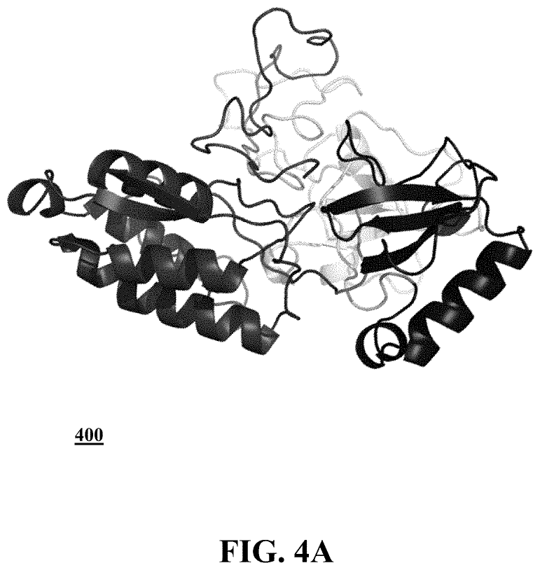

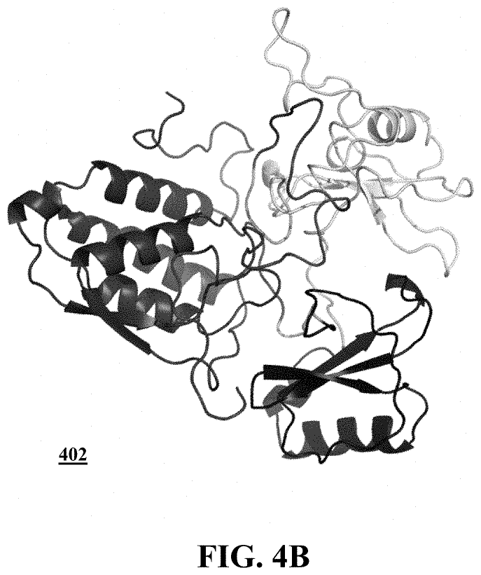

FIG. 4A illustrates a 3D structure 400 of an exemplary multi-epitope protein before 100 nanoseconds of MD simulation, consistent with one or more exemplary embodiments of the present disclosure. FIG. 4B illustrates another 3D structure 402 of an exemplary cytokine-based multi-epitope protein after 100 nanoseconds of MD simulation, consistent with one or more exemplary embodiments of the present disclosure.

### Example 3: Molecular Dynamics Simulation of the Complex of Exemplary Cytokine-Based Multi-Epitope Protein and CCR7 Receptor

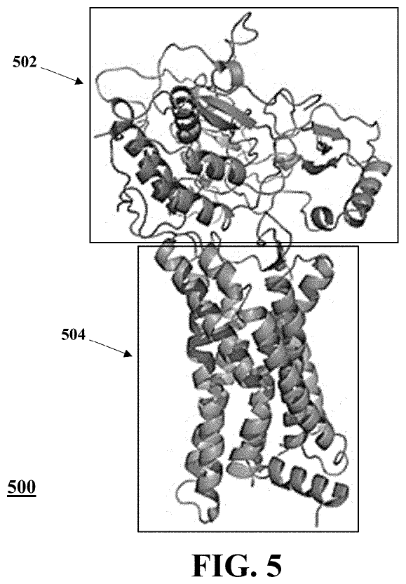

In this example, the interaction of the exemplary cytokine-based multi-epitope protein with the CCR7 receptor was examined through in-silico analyses, such as molecular docking and MD simulation. Molecular docking was done using HADDOCK software. The protein-protein docking was performed using the molecular dynamics simulation technique in a completely flexible way. Residues directly involved in connection with CCR7 are identified to limit the volume of docking calculations. The amino acids in which more than 50% water exposure are considered active amino acids and identified for the exemplary cytokine-based multi-epitope protein. TABLE 1 represents the data regarding the best cluster for the complex of CCR7 and the exemplary cytokine-based multi-epitope protein. Referring to TABLE 1, the best cluster (cluster 1) has a score of −27 and a size of 34 complexes. Also, the value of Z-Score for this cluster is equal to −2.3. FIG. 5 illustrates a molecular docking complex 500 between an exemplary cytokine-based multi-epitope protein 502 and CCR7 504, consistent with one or more exemplary embodiments of the present disclosure.

TABLE 1
Cluster data for the complex of CCR7 and the exemplary
cytokine-based multi-epitope protein

| cluster | HADDOCK score | Cluster size | Z-Score |
|---|---|---|---|
| 1 | −27 | 34 | −2.3 |

After performing molecular docking, the best molecular docking complex which is a complex with the most negative Z-score was used as an input to simulate molecular dynamics. The CCR7 protein is intermembrane; therefore, the complex of CCR7 and the exemplary cytokine-based multi-epitope protein must first be located inside the plasma membrane for MD simulation. For this purpose, the CHARMM-GUI server was used to place the complex inside the plasma membrane. Phosphatidylcholine (POPC) phospholipid was selected to make the lipid membrane. After placing the protein complex inside the POPC membrane according to the protocol in the CHARMM-GUI server, the desired output was selected for MD simulation with GROMACS 2019.6 software. At this stage, after energy minimization using the steepest descent algorithm, the system was balanced in NVT conditions in a time step of 1 femtosecond for 1 nanosecond.

Equilibration was then performed under NPT conditions in a time step of 2 femtoseconds for 4 nanoseconds. The Berendsen algorithm was used to keep the temperature constant at 310 K and the pressure at 1 atmosphere in NVT and NPT conditions. Also, during the equilibration process, a series of restrictions according to the protocols available on the CHARMM-GUI server was used to limit proteins, water, and phospholipid molecules. After the equilibration steps were completed, MD simulations were performed in the production phase for 100 nanoseconds.

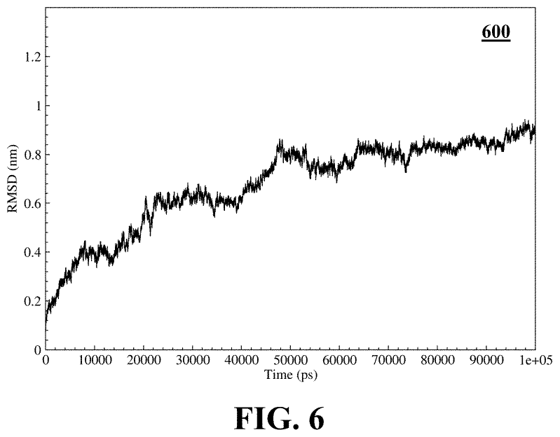

After MD simulation, the RMSD parameter was used to study the stability of the exemplary cytokine-based multi-epitope protein on the CCR7 receptor during the MD simulation. FIG. 6 illustrates diagram 600 which illustrates changes in the RMSD diagram of the exemplary cytokine-based multi-epitope protein in the CCR7 binding state during MD simulations, consistent with one or more exemplary embodiments of the present disclosure. Referring to FIG. 6, the value of RMSD shows a sharp increase at the beginning of the simulation, and after a time of 70,000 picoseconds, it reaches 0.8 nm, and after this time until the end of the simulation, it remains on the same relative stability value.

Also, the binding power of exemplary cytokine-based multi-epitope protein to the CCR7 receptor was evaluated using the molecular mechanics Poisson-Boltzmann surface area (MMPBSA) method, and the results are shown in TABLE 2.

TABLE 2
Free energy of binding an exemplary cytokine-based multi-epitope
protein to the CCR7 receptor

| Name of complex | ΔG_{vdw} | ΔG_{elec} | ΔGₛₒₗᵥ₋ₚₒₗₐᵣ | ΔGₛₒₗᵥ₋ₙₒₙₚₒₗ | ΔG_{MMPBSA} |
|---|---|---|---|---|---|
| (CCR7)-(cytokine-based | −93.344 | −94.450 | 142.388 | −15.187 | −60.592 |
| multi-epitope protein) | | | | | |

It should be noted that the more negative the bond energy, the greater the bond strength and power. As a result, referring to TABLE 2, van der Waals (ΔG_{vdw}) and electrostatic energies (ΔG_{elec}) have the same effect on the connection power and play a more significant role in the connection power in comparison with other energies. Also, the amount of polar solvent energy (ΔGₛₒₗᵥ₋ₚₒₗₐᵣ) is equal to 142.388. Moreover, the amount of free energy binding (ΔG_{MMPBSA}) of the exemplary cytokine-based multi-epitope protein to the CCR7 receptor equals −60.592 kg/mol.

### Example 4: Recombinant Production of an Exemplary Cytokine-Based Multi-Epitope Protein

In this example, the exemplary cytokine-based multi-epitope protein (SEQ ID NO: 1) was produced as a recombinant protein. Following codon optimization, the gene sequence was examined for the absence of transcriptional or translational inhibition sequences. The gene construct was then synthesized and cloned into a pET-28a vector to express the exemplary cytokine-based multi-epitope protein in E. coli. Finally, the exemplary cytokine-based multi-epitope protein was purified from the transgenic E. coli.

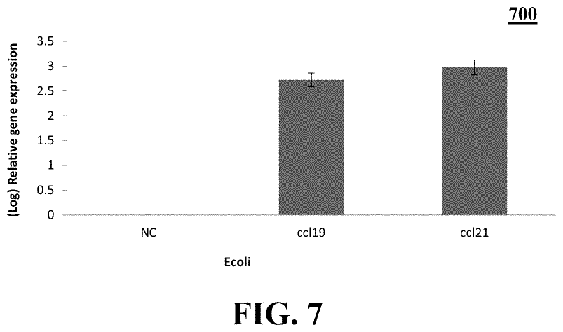

The recombinant expression of CCL21 and CCL19 epitopes of the exemplary cytokine-based multi-epitope protein in E. coli was studied by extracting the total RNA from bacteria and synthesizing complementary DNA (cDNA) through reverse transcription using oligo primer. The resulting cDNA mixtures were utilized as templates for real-time PCR. FIG. 7 illustrates diagram 700 which illustrates relative expression of CCL21 and CCL19 epitopes in E. coli compared to the beta-actin gene as a housekeeping gene using real-time PCR, consistent with one or more exemplary embodiments of the present disclosure. Referring to FIG. 7, the expression of CCL21 and CCL19 epitopes is about 2.5-fold and 3-fold higher than that of the beta-actin gene. NC group relates to results of non-transgenic E. coli as a negative control.

The recombinant protein was purified using a Ni-IDA resin affinity column, and a standard protein dot blot assay was done to measure the quantity of the purified exemplary cytokine-based multi-epitope protein. In the dot blot assay, about 10 μl of purified exemplary cytokine-based multi-epitope protein was dotted on the nitrocellulose membrane. The membrane was incubated with BSA as the blocking solution for 1 hour. After incubation, the membrane was thoroughly washed three times with phosphate-buffered saline (PBS) and incubated with conjugated anti-poly histidine tag mouse monoclonal antibody for 1 hour at 37° C., then washed three times with PBS, and finally incubated with diaminobenzidine (DAB) substrate. Also, a small amount (1 μL) of commercial CCL21 antigen containing polyhistidine tag was used as the positive control, and 10 μl of protein from the wild-type E. coli was used as the negative control.

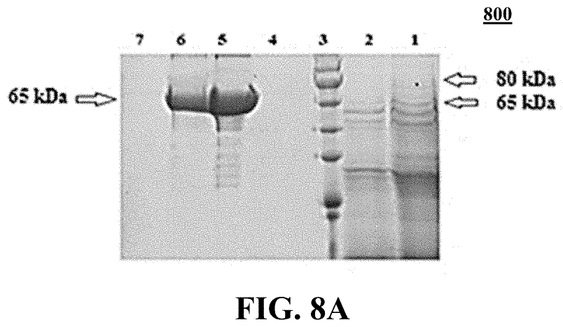

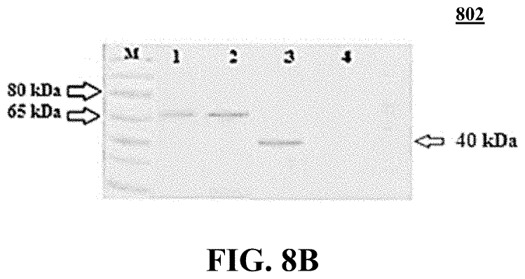

The exemplary cytokine-based multi-epitope protein expression was assessed by sodium dodecyl sulfate-polyacrylamide gel electrophoresis (SDS-PAGE), dot-blot, and Western-blot assays. FIG. 8A illustrates an image 800 which illustrates SDS-PAGE of exemplary cytokine-based multi-epitope protein, including well 1: total protein extracted from transgenic E. coli, well 2: fraction flowing from the column, well 3: protein ladder, well 4: fractions resulting from washing with 500 mM imidazole buffer solution, well 5: fractions resulting from rinsing with 100 mM imidazole buffer solution, well 6: fractions resulting from elution with 250 mM imidazole buffer solution, and well 7: Negative control (total protein extracted from non-transgenic E. coli), consistent with one or more exemplary embodiments of the present disclosure.

Referring to FIG. 8A, a 65 kDa band is observed in well 6 for fractions resulting from elution with 250 mM imidazole buffer solution, which indicates the molecular weight of the exemplary cytokine-based multi-epitope protein. On the other hand, there is no 65 kDa band in well 7 of non-transgenic E. coli, indicating no transgene expression in non-transgenic bacteria.

FIG. 8B illustrates an image 802 which illustrates Western blot results of an exemplary cytokine-based multi-epitope protein including well M: a molecular marker of protein, well 1 and well 2: exemplary cytokine-based multi-epitope protein, well 3: commercial CC121 antigen, and well 4: Negative control (total protein extracted from non-transgenic E. coli), consistent with one or more exemplary embodiments of the present disclosure. Referring to FIG. 8B, the 65 kDa band is visible in the recombinant protein extracted from the transgenic bacteria in wells 1 and 2. The band of about 40 kDa corresponds to the complete sequence of CCL21 commercial antigen as positive control is visible in well 3. Also, there is no observation of protein band in well 4 of total non-transgenic bacterial protein as the negative control.

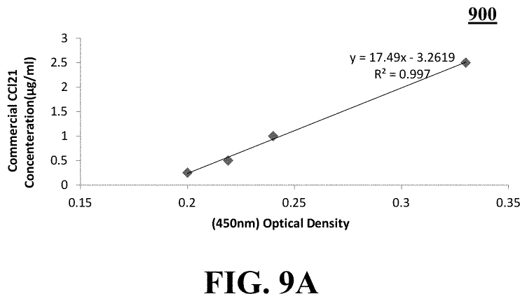

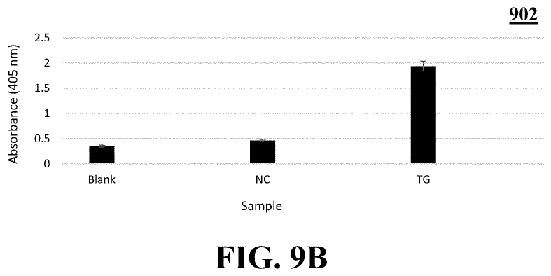

Moreover, an enzyme-linked immunosorbent assay (ELISA) was performed to determine the amount of exemplary cytokine-based multi-epitope protein using an antibody against the polyhistidine tag. FIG. 9A illustrates diagram 900 which illustrates a standard curve of commercial antigen CCL21 for determining the protein concentration based on the absorbance in the ELISA assay, consistent with one or more exemplary embodiments of the present disclosure. FIG. 9B illustrates diagram 902 which illustrates quantitative measurement of exemplary cytokine-based multi-epitope protein using ELISA, including a transgenic bacteria (TG) expressing the exemplary cytokine-based multi-epitope protein, non-transgenic bacteria as a negative control (NC), and bovine serum albumin (BSA) as a blank group, consistent with one or more exemplary embodiments of the present disclosure.

Referring to FIGS. 9A-9B, the transgenic bacteria group (TG) has the maximum and consistent expression of the exemplary cytokine-based multi-epitope protein, about 2.14% of total serum protein (TSP). Due to non-specific reactions, the observed signal in wild-type and BSA samples may be ignored.

### Example 5: Structural Analysis of an Exemplary Cytokine-Based Multi-Epitope Protein

In this example, structural features of an exemplary cytokine-based multi-epitope protein including purity were examined using matrix-assisted laser desorption/ionization time-of-flight/time-of-flight (MALDI-TOF/TOF) mass spectroscopy. At first, the purified cytokine-based multi-epitope protein was analyzed for purity by SDS-PAGE stained with G-250 Coomassie blue. Protein spots were excised from preparative stained gels, and gel slices, including the protein spots, were stained with a wash solution [100% acetonitrile and 50 mM ammonium bicarbonate (NH₄CHO₃)] for 1 hour at room temperature. The protein spot was then air-dried for 30 min at 37° C.

After that, proteins were digested using a trypsin solution (12 ng/ml trypsin in 50 mM NH₄CHO₃) by incubation for 45 minutes at 47° C. Excess trypsin solution was removed, 50 mM NH₄CHO₃ was replaced, and the gel slice was incubated overnight at 37° C. Samples were sent to the center of mass spectrometry to analyze synthetic protein by conventional ionization methods. The sequences were blasted in the National Center for Biotechnology Information (NCB) database, and their homology was examined.

MALDI-TOF/TOF results were analyzed and according to the MALDI-TOF/TOF mass spectroscopy, it was shown that CSIPAILFLPR and VQEESNDK sequences are part of human CCL21 and ILIβ with molecular weights of 1229.55 Da and 947.1 Da, respectively, whereas no bacteria sequences were detected. The average protein sequence coverage was 12%, which confirmed recombinant protein as the target protein that was correctly expressed and purified from transgenic bacteria. As a result, no protein sequence of bacteria was found in this purified protein, and just the epitope sequences of the exemplary cytokine-based multi-epitope protein were found.

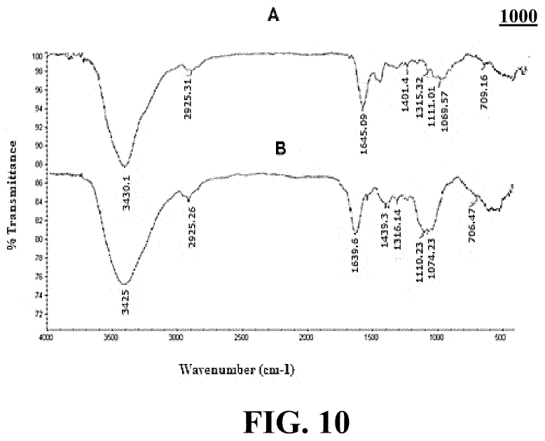

Also, the structure and post-translation modification of the exemplary cytokine-based multi-epitope protein were analyzed by infrared spectroscopy using Fourier-transform infrared spectroscopy (FTIR) spectrophotometer. FIG. 10 illustrates diagram 1000 which illustrates Fourier-transform infrared spectroscopy (FTIR) spectra of the exemplary cytokine-based multi-epitope protein (A) and commercial CCL21 antigen (B), consistent with one or more exemplary embodiments of the present disclosure.

Referring to FIG. 10, the amine, glycoside, and phospholipid factor groups were examined by studying the peaks, and similar peak positions were observed in the spectra of the exemplary cytokine-based multi-epitope protein (A) and commercial CCL21 antigen (B). There is a peak difference in the 3000 cm⁻¹-3500 cm⁻¹ region (the peak at 3430 cm⁻¹ in A and the peak at 3425 cm⁻¹ in B), which is related to the presence of OH and NH₂ groups in the commercial CCL21 antigen (B). Also, there is another peak difference in the absorption region of the CO carbonyl group (1500-2000 cm⁻¹). Assessments of the FTIR analysis and MALDI-TOF/TOF mass spectrometry displayed that the exemplary cytokine-based multi-epitope protein was correctly expressed in E. coli.

### Example 6: Effect of an Exemplary Cytokine-Based Multi-Epitope Protein on PBMC Cells

While an exemplary cytokine-based multi-epitope protein has an immunostimulatory effect, it may increase the expression of different immune genes, including CCL19, CCL21, and CCR7 in PBMC cells after incubating with an exemplary cytokine-based multi-epitope protein. In this example, expressions of CCL19, CCL21, and CCR7 genes in the PBMC cells after incubation with an exemplary cytokine-based multi-epitope protein and commercial CCL21 as a positive control were studied by real-time PCR.

First, the PBMC cells of healthy individuals and patients with colon and lung cancer were isolated and cultured. Then, the effect of the exemplary cytokine-based multi-epitope protein on the PBMC cells was evaluated by incubating the PBMC cells with a commercial CCL21 or with the exemplary cytokine-based multi-epitope protein for 48 hours, and then RNA of the PBMC cells was extracted for analyzing the gene expressions.

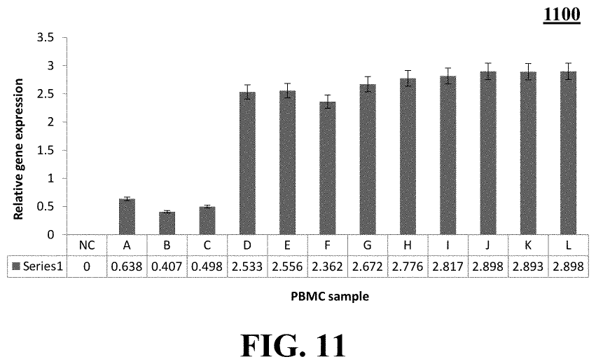

FIG. 11 illustrates diagram 1100 which illustrates relative gene expressions in different groups, including PBMC cells with only DMEM medium as a negative control (NC), expression of CCR7 (A), CCL19 (B), and CCL21 (C) in PBMC cells of cancer samples, expression of CCR7 (D), CCL19 (E), and CCL21 (F) in PBMC cells of healthy samples, expression of CCR7 (G), CCL19 (H), and CCL21 (I) in PBMC cells of cancer samples treated with cytokine-based multi-epitope protein, expression of CCR7 (J), CCL19 (K), and CCL21 (L) in PBMC cells of cancer samples treated with commercial CCL21, consistent with one or more exemplary embodiments of the present disclosure.

Referring to FIG. 11, the expressions of CCR7, CCL19, and CCL21 genes in the group treated with the exemplary cytokine-based multi-epitope protein and treated with the commercial CCL21 antigen as the positive control were higher than that of the group with only DMEM as the negative control. Also, the expression of CCR7, CCL19, and CCL21 genes in cancer patients is higher than in a treated sample; therefore, the expression of these genes may be used as a biomarker for the early detection of cancers. It should be noted that T-cells in PBMCs of individuals with CCR7⁺ receptors increase the binding affinity of CCL19 and CCL21 chemokines to the CCR7 receptors.

### Example 7: Cytotoxicity Assay of an Exemplary Cytokine-Based Multi-Epitope Protein

In this example, cytotoxicity of the exemplary cytokine-based multi-epitope protein was assessed. The MTT assay was used to determine the half-maximal inhibitory concentration (IC₅₀) of the exemplary cytokine-based multi-epitope protein on the CCR7⁺ MCF7 cancer cell line. Three days after de-freezing and culturing the cells in DMEM medium using a CO₂ incubator at 37° C., the cells were passaged and, after reaching the appropriate density of cells, the toxicity of the exemplary cytokine-based multi-epitope protein and a commercial CCL21 antigen on CCR7⁺ MCF7 cancer cell was evaluated at 24, 48, and 72 hours after incubation using MTT assay.

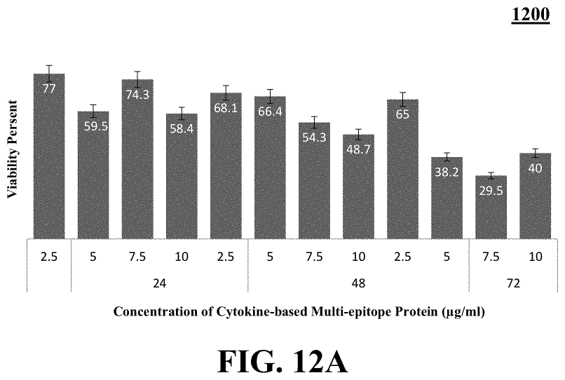

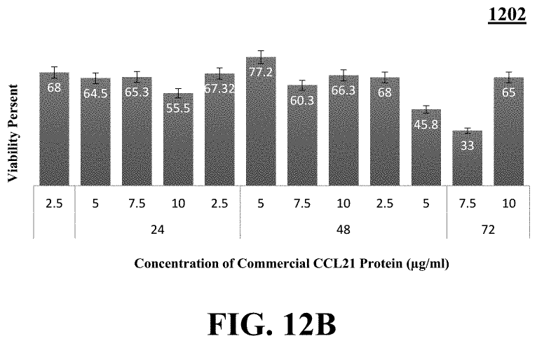

FIG. 12A illustrates diagram 1200 which illustrates an evaluation of the toxicity of the exemplary cytokine-based multi-epitope protein on CCR7⁺ MCF7 cancer cells at 24, 48, and 72 hours after incubation using the MTT assay compared to the DMEM medium as a negative control group at different concentrations of 2.5, 5, 7.5, and 10 μg/ml, consistent with one or more exemplary embodiments of the present disclosure. Referring to FIG. 12A, the survival rate of cancer cells incubated with purified recombinant protein at a 7.5 μg/ml concentration after 72 hours was 29.5% compared to the DMEM culture medium as control. Also, the IC₅₀ was 2.8 μg/ml.

FIG. 12B illustrates diagram 1202 which illustrates an evaluation of the toxicity of a commercial CCL21 antigen on MCF7 cancer cells at 24, 48, and 72 hours after incubation using the MTT assay compared to the negative control group at different concentrations of 2.5, 5, 7.5, and 10 μg/ml, consistent with one or more exemplary embodiments of the present disclosure. Referring to FIG. 12B, the results showed that at a 7.5 μg/ml concentration, the survival of cancer cells incubated with commercial CCL21 protein was 33% compared to the DMEM medium as the control group.

### Example 8: Wound Healing Assay of an Exemplary Cytokine-Based Multi-Epitope Protein

In this example, the potential activity of the exemplary cytokine-based multi-epitope protein in stimulating the proliferation and migration of cancer cells was investigated using the wound healing assay. Clinical studies discovered that MCF7 breast cancer tumors express CCR7. Therefore, to find the functional role of CCL21/CCR7, MCF7 cells were obtained and were seeded in 6-well plates at densities of 6×150 cells/well in the DMEM high glucose growth medium. After 95% confluency, cells that grew as a monolayer was scratched using a sterile pipetting tip, drawn firmly across the dish to induce in-vitro wounds. The initial width of scratches in cancer cell cultures was estimated between 700 to 800 μm. Also, PBS was used to wash the cells and remove the loosened debris.

After that 7.5 μg/ml of the exemplary cytokine-based multi-epitope protein, commercial CCL21 protein as the positive control, and DMEM medium as the negative control were added to a set of each well. For migration assay, cultures were rinsed twice using PBS solution, fixed by absolute methanol, stained by Giemsa, and inspected by a light microscope equipped with a calibrated ocular lens at 40× magnification. Images were recorded at 24, 48, and 72 hours after wounding. Cell migration rates and closure of the scratch were quantified by evaluating the changes in the wound area (pixels) using Image J software.

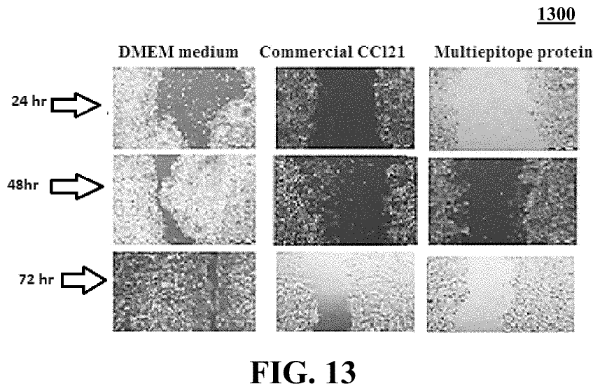

FIG. 13 illustrates image 1300 which illustrates the effect of the exemplary cytokine-based multi-epitope protein (multi-epitope protein), commercial CCL21 protein, and DMEM medium as a negative control on the migration of MCF7 cancer cells at different times (24, 48, and 72 hours after wound induction), consistent with one or more exemplary embodiments of the present disclosure. Referring to FIG. 13, migration and metastasis of cancer cells are pretty evident in negative control samples (treated with DMEM) and reach about 100% healing after 72 hours.

In contrast to the negative control samples (treated with DMEM), the migration rate is low in cells treated with the exemplary cytokine-based multi-epitope protein and commercial protein CCL21 (positive control). In the group treated with the exemplary cytokine-based multi-epitope protein, no noticeable migration of the cells was detected 24 hours after treatment, and 15% and 28% scratch closure rates occurred after 48 and 72 hours, respectively. Furthermore, while the commercial CCL21 showed 90% cancer cell migration after 72 hours, the group treated with the exemplary cytokine-based multi-epitope protein had only 35%, which shows the higher anti-metastatic effect of the exemplary cytokine-based multi-epitope protein than the commercial CCL21.

Thus, the MTT assay of EXAMPLE 7 and wound healing assay show that the exemplary cytokine-based multi-epitope protein has lethality and anti-metastasis effect on MCF7 cancer cells. In other words, the preliminary experiments showed that the exemplary cytokine-based multi-epitope protein could be a suitable choice for experimental cancer research in the future.

### Example 9: Chemotaxis Assay of an Exemplary Cytokine-Based Multi-Epitope Protein

In this example, the effect of the exemplary cytokine-based multi-epitope protein on the migration of immune cells was examined. Chemotaxis assay was used to investigate the effect of CCL21 and CCL19 epitopes of the exemplary cytokine-based multi-epitope protein on T-cell migration since chemokines are cytokines that are involved in leukocyte migration. To observe whether PBMC cells and chemoattractants, including the exemplary cytokine-based multi-epitope protein, attract each other, agarose and Boyden chamber assay was used on CCR7⁺ PBMCs. The fetal bovine serum (FBS) and a commercial CCL21 were used as positive controls

Firstly, 1% agarose was prepared in a medium composed of 50% DMEM, 10% FBS, 50% PBS, and 2 mM L-glutamine to create wells on the agarose gel. 1.6 mL of agarose solution 1% was added to each 10-mm sterile petri dish. To humidify the gel, 5 mL DMEM was added to each petri dish after 20 minutes of cooling the gel. Afterward, 5 mL FBS-free DMEM was added to the gel for 1-6 hours before performing the cell migration assay, after which three small wells with a distance of 10 mm were designed in the Petri dishes.

After that, PBMC cells were seeded at a density of 1×10⁵ cells in the middle well with 10% FBS-DMEM. After 24 hours, the medium was replaced by FBS-free DMEM. Due to their chemoattractant activity, chemokines (commercial CCL21 and the exemplary cytokine-based multi-epitope protein) were loaded in one of the neighboring wells. Also, FBS-free DMEM was loaded in other wells as the negative control. Image capture and measurements were accomplished using an inverted microscope. The cells were also stained with 4′,6-diamidino-2-phenylindole (DAPI), and the stained cells were then counted using a fluorescent microscope. Chromatin condensation and nuclear fragmentation were the criteria to confirm apoptosis.

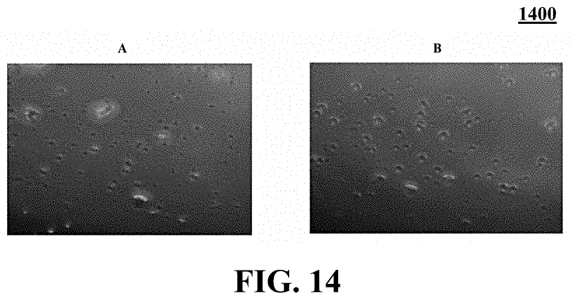

FIG. 14 illustrates image 1400 which illustrates a comparison between chemokine (CK) and chemotaxis (CT) properties: A) migration of PBMC cells to the exemplary cytokine-based multi-epitope protein containing CCL21 and CCL19 epitopes (CK), B) migration of PBMC to 10% FBS (CT), consistent with one or more exemplary embodiments of the present disclosure. Referring to FIG. 14, while there is no significant difference between chemotaxis (CT) and chemokine (CK), it may be concluded that the exemplary cytokine-based multi-epitope protein has a chemotaxis effect on the PBMC cells.

TABLE 3 represents the chemotaxis rates of different groups treated with the exemplary cytokine-based multi-epitope protein, the commercial CCL21, and FBS.

TABLE 3
Approximate number of migrating cells and percentage of chemotaxis
based on FIG. 14

| | Cells in | Cells in the | | |
|---|---|---|---|---|
| | the well of | well of non- | | |
| Adsorbent | absorbent | absorbent | Chemotaxis | Chemotaxis |
| substance | material | material | rate | percentage |
| Commercial | 170 | 17 | 153 | 90 |
| CCL21 | | | | |
| Cytokine- | 138 | 12 | 126 | 91 |
| based multi- | | | | |
| epitope protein | | | | |
| FBS 10% | 158 | 17 | 141 | 89 |

Referring to TABLE 3, the chemotaxis of each substance was assessed by counting the number of cells migrated to each well. According to the results of the exemplary cytokine-based multi-epitope protein, the number of monocyte cells moving toward the absorbent agent (138 cells) is higher than the non-absorbent substance (12 cells); as a result, chemotaxis percentage of the exemplary cytokine-based multi-epitope protein is estimated to be about 91% ((138-12)/138).

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such away. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second, and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, the inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A cytokine-based multi-epitope protein for binding to CC-chemokine receptor type 7 (CCR7)-positive cells, the cytokine-based multi-epitope comprising immunomodulatory molecules, the immunomodulatory molecules comprising:
a truncated granulocyte-macrophage colony-stimulating factor (GM-CSF); truncated chemokines, comprising a truncated CC-chemokine ligand-19 (CCL19) and a truncated CC-chemokine ligand-21 (CCL21), each of the truncated chemokines comprising:
a respective DCCL motif;
a respective putative receptor binding cleft; and
a respective putative glycosaminoglycan binding site;
a truncated interleukin 1 beta (IL-1β); and
a chemokine secretory signal peptide.

2. The cytokine-based multi-epitope protein of claim 1, wherein:
the truncated GM-CSF connected to the CCL19 through a helical linker,
the truncated CCL19 connected to the CCL21 through a furine protease-sensitive linker,
the truncated CCL21 connected to the truncated IL-1β through a cathepsin-sensitive linker, and
the truncated IL-1β connected to the chemokine secretory signal peptide directly.

3. The cytokine-based multi-epitope protein of claim 1, wherein the cytokine-based multi-epitope protein comprises SEQ ID NO: 1.

4. The cytokine-based multi-epitope protein of claim 3, wherein the cytokine-based multi-epitope protein comprises SEQ ID NO: 1 encoded by SEQ ID NO: 2.

5. The cytokine-based multi-epitope protein of claim 1, wherein the truncated GM-CSF comprises SEQ ID NO: 3.

6. The cytokine-based multi-epitope protein of claim 1, wherein the truncated CCL19 comprises SEQ ID NO: 4.

7. The cytokine-based multi-epitope protein of claim 1, wherein the truncated CCL21 comprises SEQ ID NO: 5.

8. The cytokine-based multi-epitope protein of claim 1, wherein the truncated IL-1β comprises SEQ ID NO: 6.

9. The cytokine-based multi-epitope protein of claim 1, wherein the chemokine secretory signal peptide comprises rat KC chemokine.

10. The cytokine-based multi-epitope protein of claim 9, wherein the rat chemokine KC comprises SEQ ID NO: 7.

11. The cytokine-based multi-epitope protein of claim 1, wherein the CCR7-positive cells comprise at least one of CCR7-positive breast cancer cells, CCR7-positive lung cancer cells, monocytes, T lymphocytes, B lymphocytes, natural killer (NK) cells, and dendritic cells (DCs).

12. The cytokine-based multi-epitope protein of claim 1, wherein the cytokine-based multi-epitope protein has a molecular weight between 60 kDa and 65 kDa.

13. The cytokine-based multi-epitope protein of claim 1 further comprises a purification tag, the purification tag comprising at least one of a polyhistidine tag and a glutathione S-transferase (GST) tag.

14. The cytokine-based multi-epitope protein of claim 1, wherein the cytokine-based multi-epitope protein is a hydrophilic protein with a grand average of hydropathicity index (GRAVY) of 1.25.

15. The cytokine-based multi-epitope protein of claim 1, wherein the cytokine-based multi-epitope protein is a thermostable protein with an aliphatic index of 84.57.

16. The cytokine-based multi-epitope protein of claim 1, wherein the cytokine-based multi-epitope protein is transmembrane.

17. The cytokine-based multi-epitope protein of claim 1, wherein the cytokine-based multi-epitope protein has a non-allergenicity index of more than 98%.

18. The cytokine-based multi-epitope protein of claim 1, wherein the cytokine-based multi-epitope protein is a thermostable protein with an instability index of 30.5.

19. The cytokine-based multi-epitope protein of claim 1, wherein the cytokine-based multi-epitope protein has an in-vitro half-life of less than 30 hours in mammalian reticulocytes, less than 20 hours in yeasts, and less than 10 hours in Escherichia coli cells.
